(19) United States
(12) Patent Application Publication
(10) Pub. No.: US 2025/0275671 A1
(43) Pub. Date: Sep. 4, 2025

(54) MEDICAL DEVICE ACTUATORS AND RELATED METHODS

(71) Applicant: Boston Scientific Scimed, Inc., Maple Grove, MN (US)

(72) Inventors: Scott E. Brechbiel, Arlington, MA (US); Andrew James Schaubhut, Bolton, MA (US)

(73) Assignee: Boston Scientific Scimed, Inc., Maple Grove, MN (US)

(21) Appl. No.: 19/066,877

(22) Filed: Feb. 28, 2025

Related U.S. Application Data
(60) Provisional application No. 63/648,353, filed on May 16, 2024.
(60) Provisional application No. 63/560,007, filed on Mar. 1, 2024.

(51) Int. Cl. A61B 1/00; A61B 1/015

(52) U.S. Cl. CPC A61B 1/00133; A61B 1/015

(57) ABSTRACT

A medical device may comprise a shaft extending from a proximal end to a distal end. A control wire may extend through a lumen of the shaft and to an end effector at the distal end of the shaft. The medical device may further comprise an operation portion coupled to the shaft. The operation portion may further include a housing, one or more attachment devices for coupling the medical device to another medical device, an actuation mechanism for actuating the end effector. The actuation mechanism may include a lever extending through an opening of a pivotable body within the housing. The control wire may be coupled to the pivotable body.

### CROSS-REFERENCE TO RELATED APPLICATIONS

This application claims the benefit of U.S. Provisional Patent Application Ser. No. 63/648,353, filed on May 16, 2024 and U.S. Provisional Patent Application Ser. No. 63/560,007, filed on Mar. 1, 2024, the disclosures of which are incorporated herein by reference.

### TECHNICAL FIELD

Various aspects of this disclosure relate generally to medical device actuators and related methods. In particular, aspects of this disclosure relate to actuation mechanisms for accessory devices coupled to other medical devices, among other aspects.

### BACKGROUND

During endoscopic procedures, accessory devices may be used along with scope devices. Each of the accessory device and the scope device may have one or more actuators to be activated by an operator. Conventionally, different operators have operated the accessory device and the scope device. For example, a physician may actuate, operate and/or maintain a position of a scope device (e.g., a duodenoscope) while a technician may actuate and/or operate an accessory device (e.g., a tome). The use of two operators may lead to, e.g., delayed responses, miscues, and/or increased risks during the procedure.

### SUMMARY

Each of the aspects disclosed herein may include one or more aspects of the features described in connection with any of the other disclosed aspects.

In an aspect, a medical device may comprise a shaft extending from a proximal end to a distal end. A control wire may extend through a lumen of the shaft and to an end effector at the distal end of the shaft. The medical device may further comprise an operation portion coupled to the shaft. The operation portion may further include a housing, one or more attachment devices for coupling the medical device to another medical device, an actuation mechanism for actuating the end effector. The actuation mechanism may include a lever extending through an opening of a pivotable body within the housing. The control wire may be coupled to the pivotable body.

Any of the devices, systems, or methods disclosed herein may include any of the following features, alone or in any combination. The medical device may further include a port assembly configured to couple the shaft to the operation portion. The port assembly may be rotatable relative to the operation portion. The port assembly may include two channels in fluid communication with the lumen of the shaft. One channel of the two channels may be configured to receive the control wire. Another channel of the two channels may be configured to couple to a fluid source. The pivotable body may include a protrusion configured to align with the one channel of the two channels. The housing may include a first slot on an outer surface of the housing and a second slot inside the housing. The lever may extend through the first slot and the second slot. The lever may be movable along the first slot and the second slot, and relative to the housing. The first slot and the second slot may extend parallel to one another. The pivotable body may include a channel configured to receive the control wire. The channel may include a first portion and a second portion extending approximately parallel to one another. The channel may include a third portion extending between and approximately perpendicular to the first portion and the second portion. The pivotable body may include a protrusion. The second portion of the channel may extend along the protrusion. The control wire may be fixed to the channel. The channel may include a first end and a second end. A second end of the channel may be configured to align with the lumen of the shaft. The actuation mechanism may be a first actuation mechanism configured to move the control wire in a proximal direction or a distal direction. The medical device may further include a second actuation mechanism configured to rotate the control wire. The second actuation mechanism may include a first wheel rotatably coupled to a second wheel. The control wire may extend through the first wheel and the second wheel may be coupled to a knob.

In another example, a medical device may comprise a shaft. A control wire may extend through a lumen of the shaft and to an end effector at a distal end of the shaft. The medical device may further comprise an operation portion coupled to the shaft. The operation portion may include a housing configured to be coupled to a handle of a scope device and an actuation mechanism coupled to the control wire for actuating the end effector. The actuation mechanism may include an actuator, a first arm, and a second arm movably coupled to one another.

Any of the devices, systems, and methods disclosed herein may include any of the following features, alone or in any combination. A first end of the first arm may be coupled to the actuator and a second end of the first arm may be coupled to a first end of the second arm. A second end of the second arm may be coupled to the control wire. The first arm may be configured to move in an axial direction and the second arm may be configured to rotate.

In a further aspect, a medical device may comprise a shaft including an end effector at a distal end of the shaft. A control wire may extend through the shaft and may be coupled to the end effector. The medical device may further comprise an operation portion including a housing configured to couple to a handle of another medical device and an actuation mechanism for actuating the end effector. The actuation mechanism may include a knob, a rod, and a pair of wheels. The rod may extend from the knob to a first wheel of the pair of wheels. The control wire may extend through and may be rotatably fixed to a second wheel of the pair of wheels. A rotational axis of the first wheel may be transverse to a rotational axis of the second wheel.

### BRIEF DESCRIPTION OF THE DRAWINGS

The accompanying drawings, which are incorporated in and constitute a part of this application, illustrate exemplary aspects of this disclosure and, together with the description, serve to explain the principles of the present disclosure.

FIGS. 1A and 1B illustrate an exemplary medical device, in accordance with some aspects of this disclosure.

FIGS. 2A-2D illustrate features of an exemplary medical system including the medical device of FIGS. 1A and 1B coupled to an exemplary accessory device in a first configuration (FIGS. 2A and 2B) and in a second configuration (FIGS. 2C and 2D).

FIGS. 3A-3C illustrate features of an exemplary insertion portion of the accessory device of FIGS. 2A-2D in various configurations.

FIG. 4 illustrates an exemplary alternative actuator of the accessory device of FIGS. 2A-2D.

FIGS. 5A and 5B illustrate features of an exemplary medical system including the medical device of FIGS. 1A and 1B coupled to an exemplary accessory device.

FIGS. 6A and 6B illustrate features of an exemplary medical system including the medical device of FIGS. 1A and 1B coupled to an exemplary accessory device.

FIGS. 7A and 7B illustrate features of an exemplary medical system including the medical device of FIGS. 1A and 1B coupled to an exemplary accessory device.

FIGS. 8A-8C illustrate features of an exemplary medical system including the medical device of FIGS. 1A and 1B coupled to an exemplary accessory device.

FIG. 9 illustrates an exemplary alternative actuation mechanism for an accessory device.

FIG. 10 illustrates features of an exemplary medical system including the medical device of FIGS. 1A and 1B coupled to an exemplary accessory device.

FIG. 11 illustrates features of an exemplary medical system including the medical device of FIGS. 1A and 1B coupled to an exemplary accessory device.

FIG. 12 illustrates features of an exemplary accessory device.

FIGS. 13-15 illustrate various exemplary actuators of the accessory devices of FIGS. 11 and 12.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. In various of the Figures, arrows labeled “P” and “D” illustrate proximal and distal directions.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. Ordinals such as “first,” “second,” and the like are used for convenience of referring to elements and do not imply an ordered relationship.

Although duodenoscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and/or other tools, into the subject's body. The medical devices herein may be inserted into a variety of body lumens and/or cavities, such as, for example, the urinary tract or gastrointestinal tract. It will be appreciated that, unless otherwise specified, endoscopes, bronchoscopes, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein.

Features of the medical systems herein may enable a single operator to control and/or actuate two or more medical devices either simultaneously or independently, eliminating the need for a second operator to assist with operating the devices during a medical procedure. According to some aspects of the present disclosure, the medical system may include a medical device (e.g., a duodenoscope) coupled to an accessory device. The accessory device may include one or more attachment devices to facilitate coupling the accessory device to the medical device. The accessory device may include an operation portion and an insertion portion that is sized and shaped to be received within a working lumen of the medical device. The insertion portion may be coupled to the operation portion via a rotatable port assembly including two or more channels in fluid communication with the operation portion and the insertion portion. The operation portion may include one or more actuation mechanisms for actuating a distal end of the insertion portion and/or actuating an end effector (e.g., a tome, basket, needle, snare, clip, brush, forceps, etc.) at the distal end of the insertion portion. For example, the one or more actuation mechanisms may be configured to, e.g., rotate the end effector, articulate the end effector, and/or control other aspects of the end effector.

Features of the medical systems herein may facilitate fine tune control of the medical device, the accessory device, and other medical devices (e.g., a syringe) by a single operator during a medical procedure. For example, in addition to a single operator controlling the medical device and the accessory device during a medical procedure, such as Endoscopic Retrograde Cholangiopancreatography (ERCP), it may be beneficial to have the single operator control a syringe for delivering fluid to a target site (e.g., an ampulla) to better manage pressure at the target site. Features of the medical systems herein may improve the ergonomics and/or usability of the medical device and the accessory device. For example, the operation portion of the accessory device may include, e.g., recesses, protrusions, textured surfaces, smooth surfaces, and/or markers to facilitate ease of use, among other aspects.

Reference will now be made in detail to examples of the present disclosure described above and illustrated in the accompanying drawings. Wherever possible, the same reference numbers will be used throughout the drawings to refer to the same or like parts.

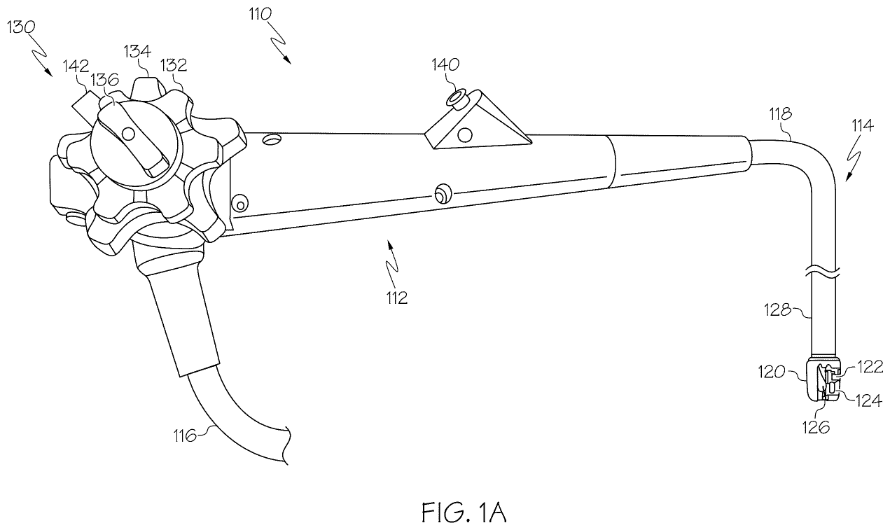

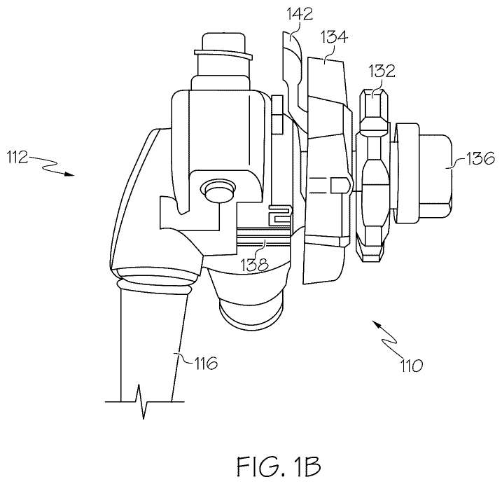

FIG. 1A depicts an exemplary medical device 110 (e.g., a duodenoscope) having a handle 112 and an insertion portion 114. FIG. 1B shows a proximal end of handle 112. Medical device 110 may also include an umbilicus 116 for purposes of connecting medical device 110 to sources of, e.g., air, water, suction, power, etc., as well as to image processing and/or viewing equipment. Insertion portion 114 may include a sheath or shaft 118 and a distal tip 120. Distal tip 120 may include an imaging device 122 (e.g., a camera) and a lighting source 124 (e.g., an LED or an optical fiber). In some examples, as shown in FIG. 1A, distal tip 120 may be side-facing (i.e., side viewing). That is, imaging device 122 and lighting source 124 may face radially outward, perpendicularly, approximately perpendicularly, or otherwise transverse to a longitudinal axis of shaft 118 and distal tip 120.

Distal tip 120 may also include an elevator 126 for changing an orientation of a medical instrument inserted in a working lumen of medical device 110. Elevator 126 may alternatively be referred to as a swing stand, pivot stand, raising base, or any suitable other term. Elevator 126 may be rotatable via, e.g., an actuation wire or another control element that extends from handle 112, through shaft 118, to elevator 126.

A distal portion of shaft 118 that is connected to distal tip 120 may have a steerable section 128. Steerable section 128 may include, e.g., an articulation joint. Shaft 118 and steerable section 128 may include a variety of structures which are known or may become known in the art.

Handle 112 may have one or more actuators/control mechanisms 130. One or more of control mechanisms 130 may provide control over steerable section 128. One or more of control mechanisms may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include a first locking mechanism 136 and a second locking mechanism 142 (e.g., knobs or levers) for preventing steering and/or braking of steerable section 128 in at least one of an up, down, left, or right direction. Handle 112 may include a control lever 138 (see FIG. 1B). Control lever 138 may raise and/or lower elevator 126, via connection between control lever 138 and an actuating wire that extends from control lever 138, through shaft 118, to elevator 126. A port 140 may allow passage of a medical instrument through port 140, into a working lumen of the medical device 110, through shaft 118, to distal tip 120.

Although FIG. 1A depicts a side-viewing medical device 110 (a device having imaging device 122 and/or lighting source 124 facing substantially radially outward), it will be appreciated that medical device 110 may alternatively be a forward-viewing device (a device having imaging device 122 and/or lighting source 124 facing substantially distally). In aspects, medical device 110 may include both a side-viewing imaging device 122 and/or lighting source 124 and a forward-facing imaging device 122 and/or lighting source 124.

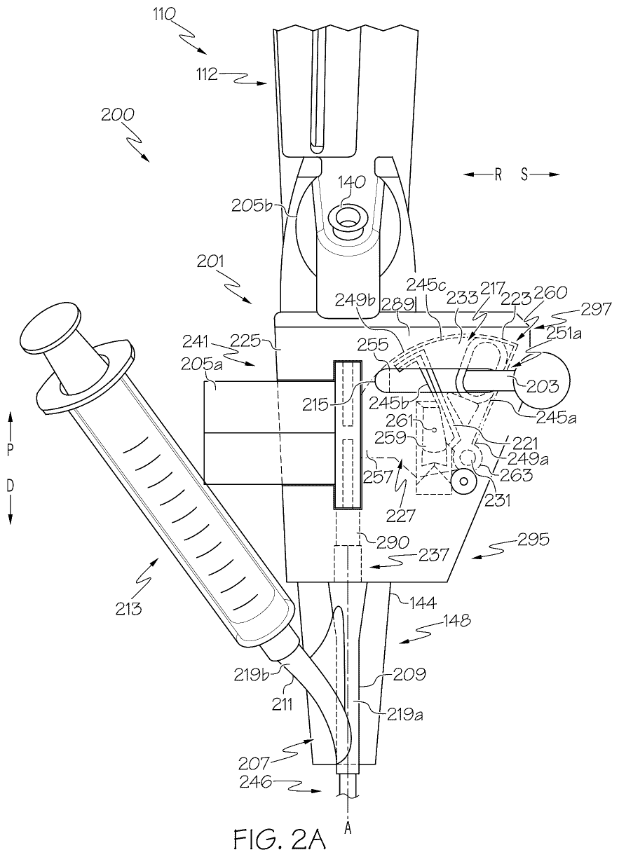

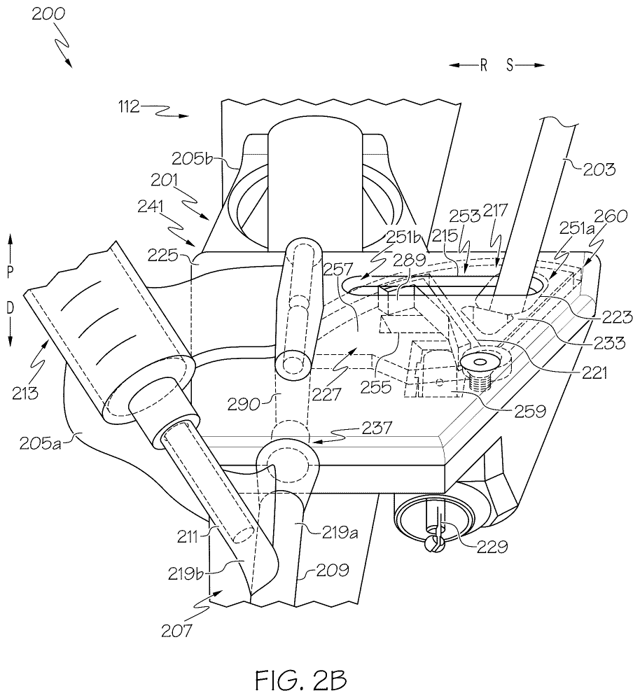

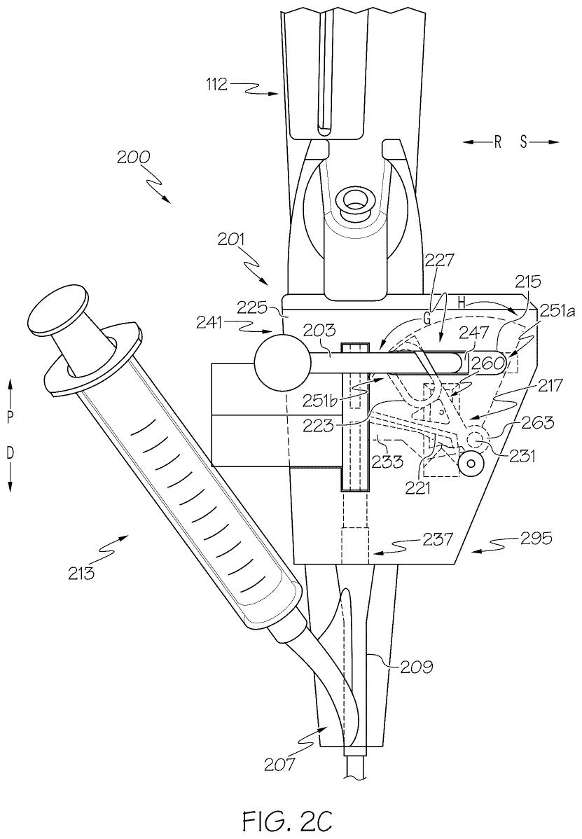

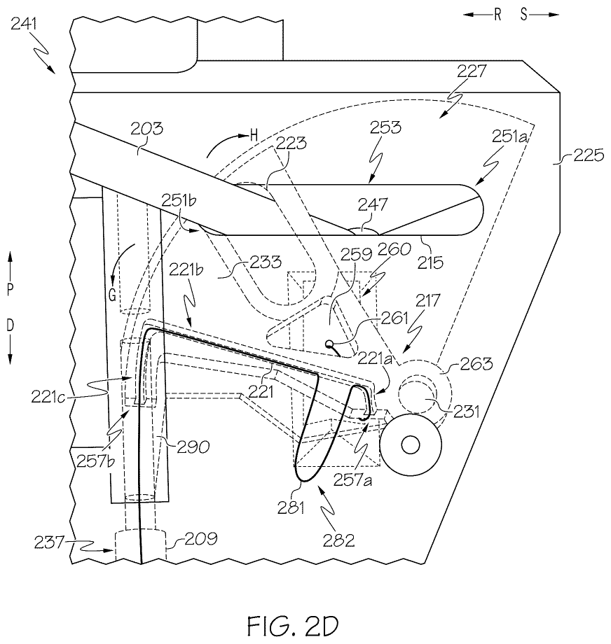

FIGS. 2A-2D illustrate internal and external features of a medical system 200 that includes medical device 110 (e.g., a first medical device) removably coupled to an accessory device 201 (e.g., a second medical device). Broken lines indicate internal features. Accessory device 201 may include an operation portion 241 having one or more attachment devices for removably coupling accessory device 201 to medical device 110. For example, operation portion 241 may include a strap 205a and a clip 205b for securing operation portion 241 to a distal portion 148 of handle 112. Strap 205a may be configured to wrap at least partially around a body 144 of handle 112. Clip 205b may be configured to clip onto sides of port 140, which may prevent rotation and/or movement of operation portion 241 relative to handle 112. Clip 205b may be integral with operation portion 241. Alternatively, clip 205b may be separate piece(s) from operation portion 241. Although strap 205a and clip 205b are described, it will be appreciated that operation portion 241 may utilize other suitable attachment devices (e.g., other straps or clips, or clamps, pincers, bands, etc.) for securing accessory device 201 to medical device 110.

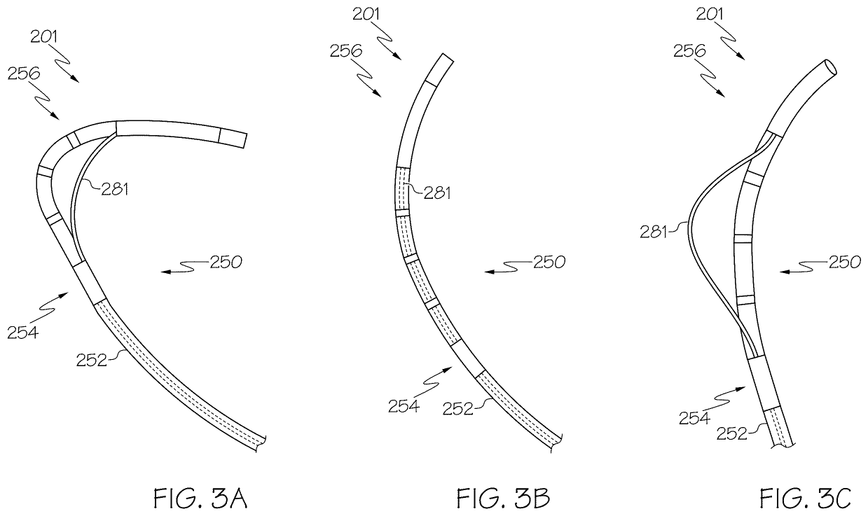

Accessory device 201 may further include a port assembly 207 (e.g., a Y-port assembly) coupled to operation portion 241. Port assembly 207 may include a first port 209 defining a first channel 219a and a second port 211 defining a second channel 219b. First port 209 may include a proximal portion 237 coupled to operation portion 241 and a distal portion 246 coupled to an insertion portion 250 of accessory device 201 (shown in FIGS. 3A-3C). For example, proximal portion 237 of first port 209 may be received within an opening formed on a housing 225 (e.g., at a distal end 295 of housing 225) of operation portion 241 to secure port assembly 207 to operation portion 241, while allowing port assembly 207 to rotate about a central longitudinal axis A of first port 209. First channel 219a may be aligned with and/or otherwise in fluid communication with one or more lumens or cavities of housing 225, e.g., a lumen 290.

Distal portion 246 of first port 209 may be coupled to a shaft 252 of insertion portion 250 (shown in FIGS. 3A-3C). Shaft 252 may extend distally from port assembly 207 and to a distal end 254 having an end effector 256. Shaft 252 may be sized and shaped to be received within port 140 and the working lumen of shaft 118 of medical device 110. In aspects, shaft 252 may define a lumen in fluid communication with first channel 219a and/or second channel 219b. Second port 211 may be configured to couple to a syringe 213 or other fluid source for delivering fluid through second channel 219b, through the lumen of shaft 252, and to a target site. Alternatively, second port 211 may allow passage of a medical instrument (e.g., a guidewire) through second channel 219b, through the lumen of shaft 252, and to a target site.

Accessory device 201 may include a control wire 281 (see, e.g., FIG. 2D) extending distally from within housing 225 (e.g., through lumen 290), through first channel 219a of first port 209, through the lumen of shaft 252, and to end effector 256 for actuating end effector 256, which will be described in greater detail below. In FIGS. 3A-3C, end effector 256 is a tome. As shown in FIGS. 3A-3C, wire 281 may include a distal end fixed to a distal end of end effector 256. Although a tome is referenced herein for illustration purposes, it will be appreciated that distal end 254 may include any suitable end effector, e.g., a basket, needle, snare, clip, brush, forceps, stent, grasper, stapler, suturing device, etc. In examples, shaft 252 may further include a braid, coil, or other structure to enable rotation of shaft 252 and/or end effector 256.

Although port assembly 207 is described as having two ports, it will be appreciated that port assembly 207 may include any number of ports, e.g., one port or three ports. For example, port assembly 207 may include first port 209 for receiving wire 281, second port 211 for receiving fluid, and a third port for receiving a guidewire. In addition, although shaft 252 is described as having one lumen, it will be appreciated that shaft 252 may include any number of lumens, e.g., two or more lumens. For example, shaft 252 may include a first lumen in communication with first channel 219a for receiving wire 281 and a second, separate lumen in communication with second channel 219b for receiving fluid.

Operation portion 241 may include an actuation mechanism 260 for actuating end effector 256. Actuation mechanism 260 may include a lever 203 (e.g., an actuator) that may be operable to pivot or rotate a pivotable member 217. Lever 203 may have a substantially straight shape. Wire 281 may extend proximally from end effector 256, through shaft 252, through first channel 219a of first port 209, through housing 225 of operation portion 241, and to actuation mechanism 260.

For example, wire 281 may be coupled to pivotable member 217. A portion of wire 281 may be fixed to pivotable member 217 such that movement of pivotable member 217 may cause movement of wire 281. Pivotable member 217 may include a body 233 positioned within a cavity 227 of housing 225. Body 233 may have a substantially triangular shape (or a shape like a sector of a circle), and may include a first edge 245a, a second edge 245b, and a substantially curved, third edge 245c. First edge 245a may have a complementary shape to a shape of a first wall 249a of cavity 227 and curved edge 245c may have a complementary shape to a shape of a curved, second wall 249b of cavity 227. Body 233 may be pivotable about a pivot point 231 in a direction G or in a direction H (shown in FIG. 2C but applicable in all of FIGS. 2A-2D). Pivot point 231 may include an axle, such as a pin, rivet, or similar structure that may be received within a ring 263 of body 233, such that pivotable member 217 is rotationally/pivotably movable about pivot point 231. Body 233 may further include a protrusion 289 extending substantially perpendicular to second edge 245b and away from first edge 245a. An outer surface of protrusion 289 may be continuous with third edge 245c. For example, a portion of third edge 245c may define a portion of protrusion 289.

Body 233 may include a channel 221 adjacent second edge 245b for receiving a portion of wire 281. As shown in FIG. 2D, channel 221 may include a first portion 221a, a second portion 221b, and a third portion 221c. First portion 221a and third portion 221c may extend approximately parallel to one another, and second portion 221b may be extend between and approximately perpendicular to first portion 221a and third portion 221c. Third portion 221c may extend along protrusion 289 of body 233. As shown in FIG. 2D, wire 281 may extend from an opening 261 of a block 259 and may enter channel 221 via an opening at a first end 257a of channel 221. Wire 281 may extend through channel 221 and exit channel 221 via an opening at a second end 257b of channel 221. At least a portion of wire 281 (e.g., a proximal portion) may be welded or otherwise adhered to channel 221. Third portion 221c of channel 221 may be configured to align with first channel 219a of first port 209. In other words, the opening at second end 257b of channel 221 may face an opening to channel 219a of first port 209. As discussed above, wire 281 may then extend through housing 225 (e.g., through lumen 290), through first channel 219a, through the lumen of shaft 252, and to end effector 256.

As shown in FIG. 2D, wire 281 may include a looped or bowed portion 282 or bend to provide strain relief during movement of pivotable member 217 and/or wire 281. Looped or bowed portion 282 may not be fixed within channel 221. Alternatively, the looped or bowed portion 282 or bend may be omitted.

Body 233 of pivotable member 217 may further include an opening 223 for receiving lever 203. Lever 203 may extend through a first slot 215 formed on an outer surface of housing 225, through cavity 227, and through a second slot 255 (see FIG. 2B) within housing 225. For example, second slot 255 may be positioned below and aligned with first slot 215 and may be formed on an interior surface 257 of housing 225. First and second slots 215, 255 may be at a proximal end 297 of housing 225, and may extend approximately parallel to one another and approximately perpendicular to central longitudinal axis A of first port 209. In other words, first and second slots 215, 255 may extend approximately perpendicular to a longitudinal axis of handle 112 when accessory device 201 is coupled to medical device 110. Slots 215 and 255 may be linear.

Lever 203 may be pivotable about an axle 247 (FIGS. 2C and 2D) and movable along/through first and second slots 215, 255 in a direction R or a direction S (i.e., in directions perpendicular to P/D directions) relative to housing 225. Slots 215 and 255 may constrain lever 203 to linear motion through slots 215 and 255. Because lever 203 extends through opening 223, lever 203 may exert forces on walls of opening 223 to cause pivotable member 217 to move. Movement of lever 203 in direction R may pivot pivotable member 217 in direction G, e.g., such that protrusion 289 moves toward distal end 295 of housing 225. Movement of lever 203 in direction S may pivot pivotable member 217 in direction H, e.g., such that protrusion 289 moves away from distal end 295 of housing 225. Opening 223 may have a width that is approximately the same as a width of lever 203 and a length that is greater than a length of lever 203, such that lever 203 may move along a length of opening 223 as pivotable member 217 pivots.

In some aspects, wire 281 may be coupled to an electrical socket 229 (shown in FIG. 2B). For example, a proximal end of wire 281 may be received within opening 261 formed on block 259 coupled to electrical socket 229. Operation portion 241 may be connected to an electrical supply through electrical socket 229 to electrify wire 281, which may provide, for example, electricity to end effector 256 for electrical cautery or cutting of tissue.

FIGS. 2A and 2B illustrate accessory device 201 in a first configuration in which lever 203 may be positioned at a first end 251a of slot 215. In the first configuration, protrusion 289 of pivotable member 217 may be positioned away from distal end 295 of housing 225. For example, first edge 245a of pivotable member 217 may contact or be adjacent to first wall 249a of cavity 227. In the first configuration, wire 281 may be in a pulled/tensioned state (i.e., tension may be applied to wire 281). In other words, wire 281 may be in a proximal configuration. In an example in which end effector 256 is a tome, end effector 256 may be in an actuated state (e.g., a forward bend), as shown in FIG. 3A. In other examples, the first configuration of FIGS. 2A and 2B may correspond to actuated states of other types of end effectors 256 or may correspond to an unactuated state of other types of end effectors 256.

Lever 203 may be moved in direction R to transition accessory device 201 to a second configuration in which lever 203 may be positioned at a second end 251b of slot 215, as shown in FIGS. 2C and 2D. Movement of lever 203 in direction R along slots 215, 255 may cause pivotable member 217 to pivot about pivot point 231 in direction G, such that protrusion 289 moves toward distal end 295 of housing 225. In this configuration, wire 281 may be moved in a distal direction D (e.g., wire 281 may be relaxed). As shown in FIG. 2D, in the second configuration, third portion 221c of channel 221 may be aligned with lumen 290 and first port 209. In the second configuration, in an example in which end effector 256 is a tome, end effector 256 may be in an unactuated or relaxed state, as shown in FIG. 3B. In other examples, the second configuration of FIGS. 2C and 2D may correspond to unactuated states of other types of end effectors 256 or may correspond to an actuated state of other types of end effectors 256.

To transition accessory device 201 back to the first configuration, lever 203 may be moved in direction S along slots 215, 255 to pivot pivotable member 217 about pivot point 231 in direction H. Movement of pivotable member 217 in direction H may pull wire 281 proximally (in a proximal direction P), to actuate end effector 256 or return end effector 256 to an unactuated state.

Alternatively, features of accessory device 201 may be configured such that positioning of lever 203 at an intermediate portion 253 of slot 215 (shown in FIG. 2B) may transition end effector 256 to an unactuated or relaxed state (such as that shown in FIG. 3B for a tome). In such examples, positioning lever 203 at first end 251a or second end 251b may cause end effector 256 to transition to a first or a second actuated state. For example, in an example in which end effector 256 is a tome, positioning of lever 203 at second end 251b of slot 215 may transition end effector 256 to a second actuated state (i.e., a reverse bend), as shown in FIG. 3C.

During an exemplary medical procedure, an operator may couple accessory device 201 to medical device 110 using strap 205a and clip 205b. The operator may then couple syringe 213 to second port 211 of port assembly 207. The operator may grasp onto handle 112 of medical device 110 with, e.g., their left hand. The operator may navigate shaft 118 of medical device 110 to a position proximate a target site, e.g., a major duodenal papilla. The operator may then insert shaft 252 of accessory device 201 into port 140 of medical device 110 and extend shaft 252 through the working lumen of medical device 110 using, e.g., their right hand. Shaft 252 of accessory device 201, including end effector 256, may exit the working lumen of medical device 110. The operator may use their left hand (or right hand) to control lever 138 to raise elevator 126 and angle end effector 256 toward the papilla. The operator may use their right hand (or left hand) to control lever 203 of accessory device 201 to actuate end effector 256 to cannulate the papilla or perform another medical procedure. The operator may also use their right (or left) hand to operate syringe 213 for delivering fluid during the procedure. Optionally, the operator may rotate port assembly 207 relative to operation portion 241 to move syringe 213 out of the way if, e.g., syringe 213 is impeding the operator's access to lever 203 or to rotate shaft 252. Thus, a single operator may use medical device 110 and use/actuate accessory device 201.

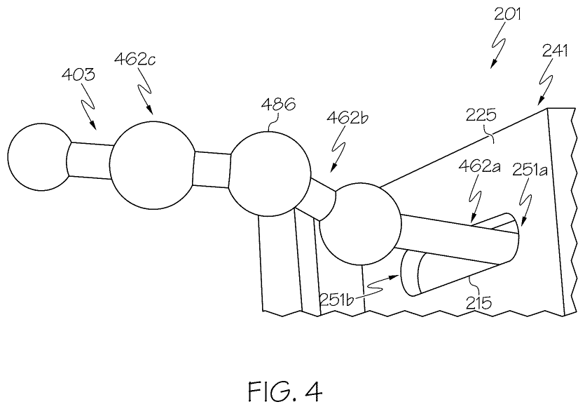

FIG. 4 illustrates an exemplary lever 403 for use with accessory device 201 in place of lever 203. Lever 403 may include angled portions and/or other ergonomic features to facilitate ease of use. For example, lever 403 may include a first portion 462a, a second portion 462b, and a third portion 462c. First portion 462a may extend linearly from housing 225 and out of slot 215, second portion 462b may extend from and be angled relative to first portion 462a and toward first end 251a of slot 215, and third portion 462c may extend from and be angled relative to second portion 462b and toward second end 251b of slot 215. Angled portions 462b, 462c may allow for an operator to more easily move lever 403 along slot 215 to actuate end effector 256. In further alternatives, a bent lever or a T-lever may be used in place of levers 203, 403. Lever 403 may include a plurality of rounded, protruding portions 486 that may assist an operator with gripping lever 403 or using lever 403 with a single finger.

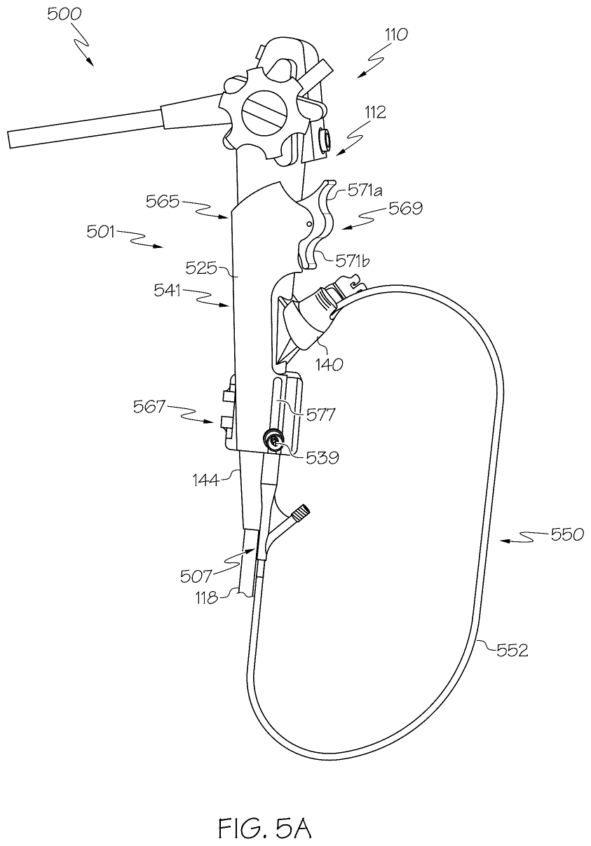

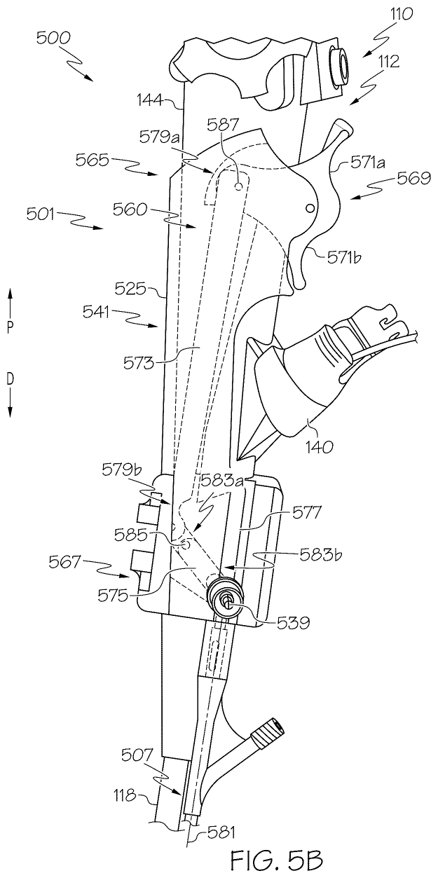

FIGS. 5A and 5B illustrate features of an exemplary medical system 500 that includes medical device 110 coupled to an exemplary accessory device 501, which may have any of the features of accessory device 201, unless otherwise specified herein. Accessory device 501 may include a port assembly 507 having any of the features of port assembly 207 and an insertion portion 550 having any of the features of insertion portion 250.

Accessory device 501 may include an operation portion 541 having a housing 525. Housing 525 may include or be coupled to one more attachment devices for coupling operation portion 541 to a handle 112 of medical device 110. For example, housing 525 may be coupled to body 144 of handle 112 via a snap-fit connection. For example, one or more walls of housing 525 may define a recess for receiving body 144 and for securing body 144 with an interference fit. Alternatively, housing 525 may include straps, bands, clips, jaws, or pincers, as discussed above for system 200.

Operation portion 541 may include an actuation mechanism 560 for actuating an end effector, e.g., end effector 256, at a distal end of a shaft 552 of insertion portion 550. For example, a control wire 581 (shown in FIG. 5B) may extend proximally from end effector 256, through shaft 552, through port assembly 507, and to actuation mechanism 560.

As shown particularly in FIG. 5B, actuation mechanism 560 may include an actuator 569, a first arm 573, a pin 585, a second arm 575, and a connection pin 539 movably coupled to one another. Although the term “connection pin” 539 is used herein, it will be appreciated that connection pin 539 may include a bar, a rod, or any other suitable structure. Actuator 569 may be positioned at a proximal portion 565 of housing 525.

Actuator 569 may be, e.g., a toggle or a lever, and may include a first contact element 571a (e.g., a contact surface) and a second contact element 571b (e.g., a contact surface). First and second contact elements 571a, 571b may include concave surfaces for ease of use. For example, an operator may apply a force on first contact element 571a to move first contact element 571a toward body 144 and second contact element 571b away from body 144. Conversely, an operator may apply a force on second contact element 571b to move second contact element 571b toward body 144 and first contact element 571a away from body 144. Alternatively, actuator 569 may be or may include a switch, button, knob, a lever, and/or other structure.

As shown in FIG. 5B, actuator 569 may be coupled to a first end 579a of first arm 573 via e.g., a hinge 587. In examples, first end 579a may be pivotably/rotationally coupled to actuator 569. First arm 573 may extend distally within housing 525 to second arm 575 within and at a distal portion 567 of housing 525. A second end 579b of first arm 573 may be coupled to a first end 583a of second arm 575. First arm 573 may be movable in generally proximal and distal directions (e.g., in an axial direction), and second arm 575 may be pivotable about pin 585. A second end 583b of second arm 575 may be pivotably/rotationally coupled to a connection pin 539.

Connection pin 539 may be movable along a channel 577 formed at distal portion 567 of housing 525. Channel 577 may extend approximately parallel to a longitudinal axis of housing 525, such that connection pin 539 may move in proximal/distal directions along channel 577. Connection pin 539 may be coupled to a proximal end of wire 581, such that movement of connection pin 539 in proximal and distal directions may cause movement of wire 581 in the corresponding proximal and distal directions to actuate end effector 256. Connection pin 539 may also be configured to couple to an electrical supply to electrify wire 581.

FIGS. 5A and 5B illustrate a first configuration of accessory device 501 in which wire 581 is in a distal configuration (e.g., an actuated configuration of the tome shown in FIGS. 3A-3C). In use, an operator may grasp onto, e.g., a proximal portion of handle 112 using their left hand. The operator may then place, e.g., their ring finger on first contact element 571a and their pinky finger on second contact element 571b. The operator may apply a force onto first contact element 571a using their ring finger to move first contact element 571a toward body 144 of handle 112 transitioning accessory device 501 to a second configuration. Movement of first contact element 571a toward body 144 of handle 112 may cause first arm 573 to move distally. Distal movement of first arm 573 may cause second arm 575 to pivot about pin 585, e.g., counterclockwise in the frame of reference shown in FIG. 5B. Such movement of second arm 575 may cause connection pin 539 to move proximally along channel 577, which in turn may move or pull wire 581 proximally to actuate end effector 256 (or to transition end effector 256 to an unactuated state for a device that is actuated by moving pull wire 581 distally).

The operator may apply a force onto second contact element 571b using their pinky finger to transition accessory device 501 back to the first configuration. Movement of second contact element 571b toward body 144 of handle 112 may cause first arm 573 to move proximally. Proximal movement of first arm 573 may cause second arm 575 to pivot about pin 585 in an opposite direction to that described above, e.g., clockwise in the frame of reference shown in FIG. 5B. Such movement of second arm 575 may cause connection pin 539 to move distally along channel 577, which in turn may move or push wire 581 distally to transition end effector 256 to an unactuated or relaxed state (or to an actuated state of a medical device that is unactuated by moving wire 581 proximally).

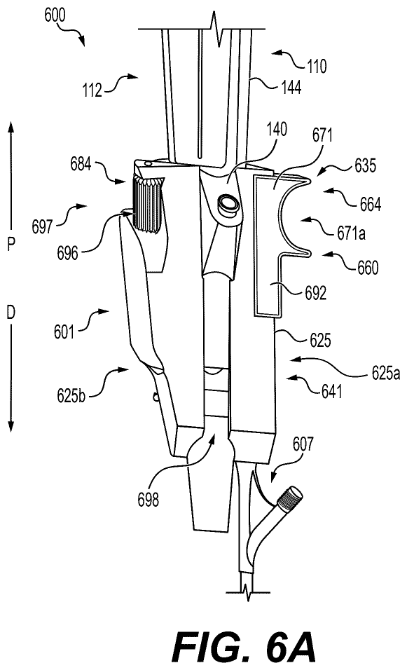

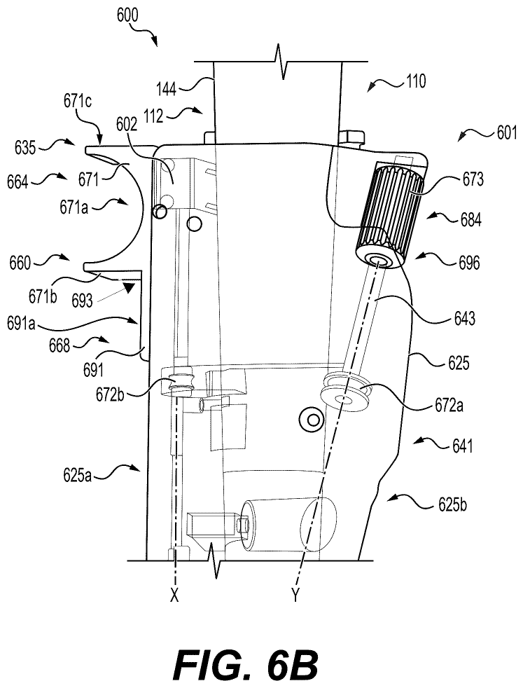

FIGS. 6A and 6B illustrate internal and external features of an exemplary medical system 600 that includes medical device 110 coupled to an exemplary accessory device 601. Accessory device 601 may have any of the features of accessory devices 201, 501, unless otherwise specified herein. FIG. 6A illustrates a front view of medical system 600 and FIG. 6B illustrates a back view of medical system 600. Accessory device 601 may include a port assembly 607 having any of the features of port assemblies 207, 507. Port assembly 607 may be configured to couple to an insertion portion (not shown) having any of the features of insertion portions 250, 550. For example, the insertion portion of accessory device 601 may include a shaft (not shown) extending distally from port assembly 607 and a distal end of the shaft may include an end effector (not shown), e.g., end effector 256. A control wire (not shown for clarity of illustration in FIGS. 6A and 6B but having any of the properties of control wires 281, 581 may extend proximally from end effector 256 (or another end effector), through the shaft, through port assembly 607, and to an operation portion 641 of accessory device 601. The control wire may be coupled, directly or indirectly, to a first actuation mechanism 660 and a second actuation mechanism 696 of operation portion 641 for actuating end effector 256 (or an alternative end effector), which will be discussed in greater detail below.

Operation portion 641 may include a housing 625 having one more attachment devices for coupling operation portion 641 to handle 112 of medical device 110. For example, housing 625 may be coupled to body 144 of handle 112 via a snap-fit connection. As shown in FIG. 6A, one or more walls of housing 625 may define a recess 698 on a front side of housing 625 for receiving body 144 and for securing body 144 with an interference fit. Additionally or alternatively, housing 625 may include straps, bands, clips, jaws, or pincers, as discussed above for system 200, to secure operation portion 641 to handle 112.

As mentioned above, operation portion 641 may include first actuation mechanism 660 and second actuation mechanism 696 coupled to the control wire. First actuation mechanism 660 may be configured to move the control wire proximally and distally, and second actuation mechanism 696 may be configured to rotate the control wire. The control wire or portions of the control wire (e.g., proximal portions) may have any noncircular shape (e.g., a ribbon shape, a rectangle shape, etc.) to rotatably fix (e.g., key) the control wire to a second wheel 672b (FIG. 6B) of second actuation mechanism 696, which will be discussed in greater detail below. In some examples, only a portion of the control wire that extends through second wheel 672b may have a non-round shape.

First actuation mechanism 660 may include a slider 635 (e.g., a first actuator) movable proximally and distally along a slot formed on an outer surface on a first lateral side 625a of housing 625. A proximal portion 664 of slider 635 may include a contact element 671 (e.g., a body) having an outer, concave surface 671a extending between first and second outer, planar surfaces 671b, 671c (labeled in FIG. 6B). Concave surface 671a may face away from housing 625, and first and second planar surfaces 671b, 671c may extend perpendicular to a longitudinal axis of housing 625.

As more clearly shown in FIG. 6B, a distal portion 668 of slider 635 may include a first wall 691 recessed from contact element 671 and extending parallel to a longitudinal axis of housing 625. Outer, planar surface 671b of contact element 671 and an outer, planar surface 691a of first wall 691 may define a corner 693 of slider 635. An inner surface of contact element 671 and an inner surface of first wall 691 may form a continuous first inner surface of slider 635 extending parallel to a longitudinal axis of housing 625 and slidable along the outer surface on first lateral side 625a of housing 625 and/or the slot of housing 625. The shape of slider 635 discussed above may allow an operator to actuate slider 635 without looking at slider 635 and/or retain a finger of the operator along concave surface 671a. Additionally or alternatively, an operator may rest his or her finger on planar surface 671b when operating slider 635. The shape of slider 635 may thus offer multiple ergonomic options for an operator. However, the shape of slider 635 described above is merely exemplary.

As shown in FIG. 6A, slider 635 may include a second wall 692 disposed on a front side of housing 625. Second wall 692 may extend from a proximalmost end of slider 635 to a distalmost end of slider 635, and may extend approximately perpendicularly from contact element 671 and first wall 691. Second wall 692 may define a second inner surface of slider 635 slidable along an outer surface on the back side of housing 625 (e.g., an outer surface that extends approximately perpendicularly from the outer surface on first side 625a of housing 625).

Alternatively, second wall 692 may be omitted from slider 635. As shown in FIG. 6B, slider 635 may further include a projection 602 coupled to the inner surface of contact element 671. Projection 602 may movable within housing 625 and along the slot of housing 625. Projection 602 may be coupled to and/or may extend from a portion of the inner surface of contact element 671 adjacent to the proximalmost end of slider 635. Alternatively, projection 602 may be coupled to and/or may extend from any portion of the inner surface of contact element 671 and/or first wall 691. The control wire may be coupled to or fixed to protrusion 602, such that movement of slider 635 proximally or distally along the slot of housing 625 may move the control wire proximally or distally.

Second actuation mechanism 696 may include a knob 684 (e.g., a second actuator) disposed on a second lateral side 625b of housing 625 and at a proximal end 697 of housing 625. For example, knob 684 may be disposed within a recess of housing 625. Knob 684 may include a plurality of indentations 673 or other gripping features that may assist an operator with rotating knob 684 with, e.g., their thumb. As shown in FIG. 6B, second actuation mechanism 696 may further include a rod 643, and first and second wheels 672a, 672b disposed within housing 625 and on opposite sides of housing 625. For example, first wheel 672a may be positioned closer to second lateral side 625b and second wheel 672b may be positioned closer to first lateral side 625a. A diameter of first wheel 672a may be greater than a diameter of second wheel 672b. Alternatively, a diameter of first wheel 672a may have a same diameter as second wheel 672b or second wheel 672b may have a greater diameter.

Portions of rod 643 may extend through knob 684 and remaining portions of rod 643 may extend within housing 625. Rod 643 may extend distally through knob 684 and towards a central longitudinal axis of housing 625 to first wheel 672a. First and second ends of rod 643 may be fixed to knob 684 and to first wheel 672a, respectively. Thus, rotation of knob 684 about a rotation axis Y in a direction (e.g., clockwise or counterclockwise) may rotate rod 643 and first wheel 672a about rotation axis Y in the same direction. Axis Y may be transverse (not parallel to) the central longitudinal axis of housing 625.

Second wheel 672b may be distal to and aligned with protrusion 602 of slider 635, and substantially aligned with first wheel 672a along an axis extending approximately perpendicularly to a longitudinal axis of housing 625. First wheel 672a and second wheel 672b may be coupled to one another via, e.g., a belt or chain (not shown but having features of a belt 878, discussed below) extending from first wheel 672a to second wheel 672b, such that rotation of first wheel 672a about rotation axis Y in a direction (e.g., clockwise or counterclockwise) rotates second wheel 672b about a rotation axis X. As shown in FIG. 6B, rotational axis Y may extend transversely (not parallel) to rotational axis X. In other words, axis Y may have a non-zero angle with respect to axis X. Alternatively, knob 684, rod 643, and wheel 672a may be arranged such that rotational axis Y may extend parallel to or approximately parallel to rotational axis X.

The control wire may extend distally from protrusion 602 and through an opening of second wheel 672b (e.g., an opening having any of the properties of opening 774 below). As mentioned above, the control wire (or portions of the control wire) may have any noncircular shape. A size and a shape of the opening of second wheel 672b may be keyed to/complementary to a size and shape of the control wire, which may allow for the control wire to move proximally or distally through the opening of second wheel 672b, but may prevent rotation of the control wire relative to second wheel 672b. In other words, the control wire may be freely movable proximally or distally (e.g., in a longitudinal direction) within the opening of second wheel 672b and relative to second wheel 672b due to proximal/distal movement of slider 635. However, the control wire may be rotatably fixed to second wheel 672b. For example, rotation of knob 684 and thus rotation of second wheel 672b about rotation axis X in a direction (e.g., clockwise or counterclockwise), rotates the control wire about rotation axis X in the same direction.

FIGS. 6A and 6B illustrate slider 635 in a proximal position in which the control wire may be in a pulled/tensioned state (e.g., a proximal configuration). In an example in which end effector 256 is a tome, end effector 256 may be in an actuated state (e.g., a forward bend), as shown in FIG. 3A, when slider 635 is in the proximal position. To transition the control wire to a relaxed state (e.g., a distal configuration), an operator may place their ring finger or other finger on concave surface 671a of contact element 671 and push downward/distally on contact element 671 to move slider 635 distally along the slot of housing 625 to a distal position. In an example in which end effector 256 is a tome, end effector 256 may be in an unactuated or relaxed state, as shown in FIG. 3B, when slider 635 is in the distal position. The operator may use their thumb of the same hand to rotate knob 684 to rotate the control wire/end effector 256.

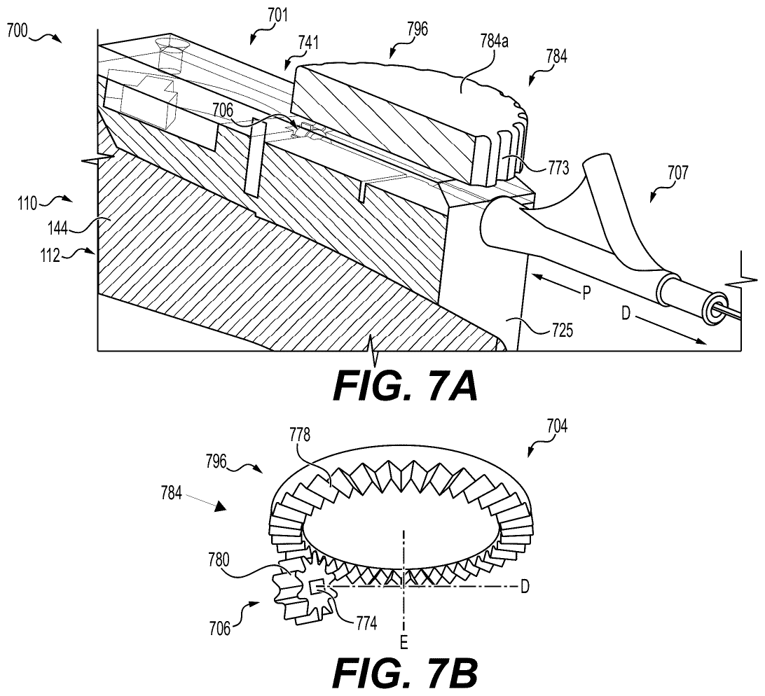

FIGS. 7A and 7B illustrate features of an exemplary medical system 700 that includes medical device 110 coupled to an exemplary accessory device 701. Accessory device 701 may have any of the features of accessory devices 201, 501, 601, unless otherwise specified herein. FIG. 7A illustrates a cross-sectional view of a portion of medical system 700. Accessory device 701 may include a port assembly 707 having any of the features of port assemblies 207, 507, 607. Port assembly 707 may be configured to couple to an insertion portion (not shown) having any of the features of insertion portions 250, 550. For example, the insertion portion of accessory device 701 may include a shaft (not shown) extending distally from port assembly 707 and a distal end of the shaft may include an end effector (not shown), e.g., end effector 256. A control wire (not shown but having the features of any of control wires 281, 581) may extend proximally from end effector 256, through the shaft, through port assembly 707, and to an operation portion 741 of accessory device 701. The control wire may be coupled to a first actuation mechanism (not shown in FIGS. 7A and 7B) of operation portion 741 having any of the features of actuation mechanisms 260, 560, 660 for controlling proximal and distal movement of the control wire to control aspects of end effector 256, as described above.

The control wire may be further coupled to a second actuation mechanism 796 of operation portion 741. Second actuation mechanism 796 may be configured to rotate the control wire and thus rotate end effector 256. Second actuation mechanism 796 may include a knob 784 disposed on a side of a housing 725 of operation portion 741. A plurality of indentations 773 or other gripping features may be disposed around a circumference/perimeter of knob 784 to assist an operator with rotating knob 784. Knob 784 may function as a gear 704. An outer side of knob 784 may include a planar surface 784a and an inner side of knob 784 may form first gear 704 (shown in FIG. 7B). For example, knob 784/gear 704 may form a crown gear, having teeth 778 facing/extending inward (i.e., a direction approximately perpendicular to planar surface 784a, toward a central longitudinal axis of device 110). Teeth 778 may be directly molded onto a surface on the inner side of knob 784.

As more clearly shown in FIG. 7B, teeth 778 of first gear 704 may be configured to engage with teeth 780 of a second gear 706, e.g., a pinion gear, disposed within housing 725. Engagement of teeth 778 and teeth 780 may be configured to rotate second gear 706 upon rotation of knob 784. Second gear 706 may be oriented approximately perpendicular to first gear 704, such that a rotational axis D of second gear 706 may be approximately perpendicular to a rotational axis E of knob 784/first gear 704. Although FIG. 7B does not show all of the features of knob 784 (e.g., indentations 773) for ease of illustration, it will be appreciated that any of such features may be disposed on knob 784 of FIG. 7B.

Similar to the control wire of FIGS. 6A and 6B, the control wire of FIGS. 7A and 7B or a portion thereof may also have any noncircular shape. The control wire may extend through a central opening 774 of second gear 706. A size and a shape of central opening 774 of second gear 706 may be keyed to/complementary to a size and shape of the control wire, which may allow for the control wire to move proximally or distally through central opening 774 of second gear 706, but may prevent rotation of the control wire relative to second gear 706. For example, the control wire and central opening 774 of second gear 706 may each have a rectangle shape; however, such a shape is merely exemplary and any suitable non-round shape may be utilized (e.g., oval, elongate, polygonal, etc.). The control wire may be rotatably fixed relative to second gear 706 such that rotation of second gear in a direction (e.g., clockwise or counterclockwise) about rotational axis D rotates the control wire in the same direction about rotational axis D.

In use, an operator may, e.g., rotate knob 784 clockwise about rotational axis E, which in turn rotates second gear 706 and the control wire clockwise about rotational axis D, to rotate end effector 256 to a first position. Axis E may be approximately perpendicular to a central longitudinal axis of medical device 110, and rotational axis D may be approximately parallel to the central longitudinal axis of medical device 110. The operator may then, e.g., rotate knob 784 counterclockwise about rotational axis E, which in turn rotates second gear 706 and the control wire counterclockwise about rotational axis D, to rotate end effector 256 to a second position.

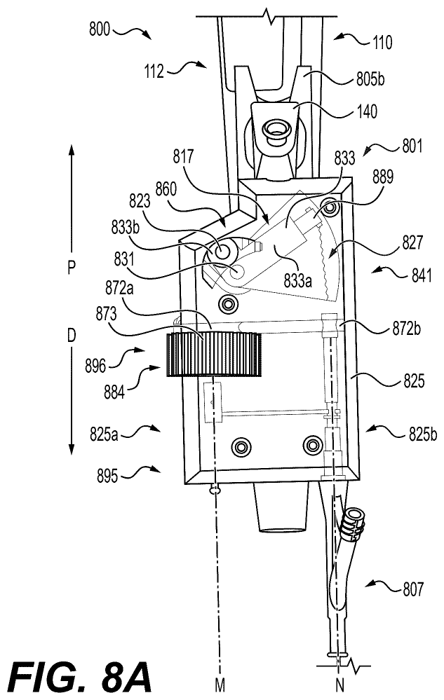

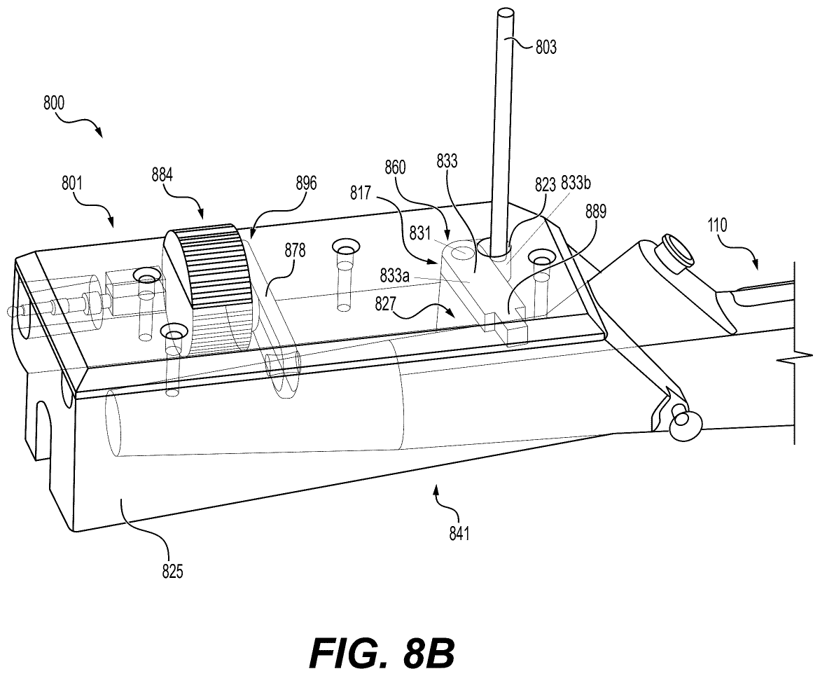

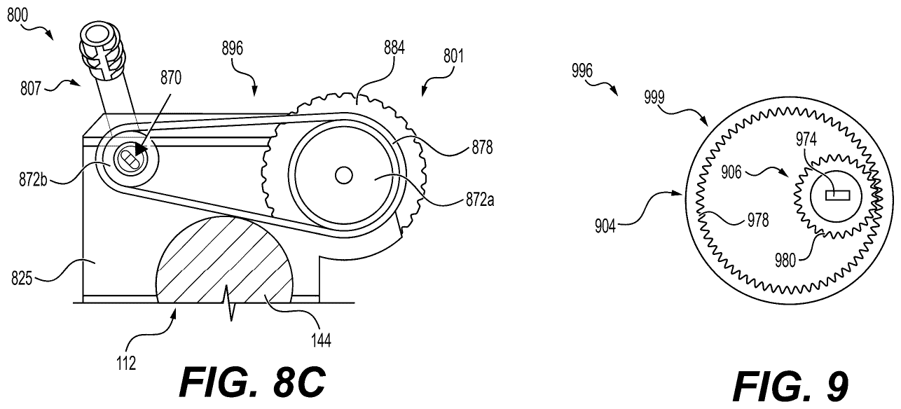

FIGS. 8A-8C illustrate internal and external features of an exemplary medical system 800 that includes medical device 110 coupled to an exemplary accessory device 801. Accessory device 801 may have any of the features of accessory devices 201, 501, 601, 701, unless otherwise specified herein. Accessory device 801 may include a port assembly 807 having any of the features of port assemblies 207, 507, 607, 707. Port assembly 807 may be configured to couple to an insertion portion (not shown) having any of the features of insertion portions 250, 550. For example, the insertion portion of accessory device 801 may include a shaft (not shown) extending distally from port assembly 807 and a distal end of the shaft may include an end effector (not shown), e.g., end effector 256. A control wire (not shown but having any of the features of control wires 281, 581) may extend proximally from end effector 256, through the shaft, through port assembly 807, and to an operation portion 841 of accessory device 801.

The control wire may be coupled (directly or indirectly) to a first actuation mechanism 860 and a second actuation mechanism 896 of operation portion 841 for actuating end effector 256, which will be discussed in greater detail below. Operation portion 841 may include a housing 825 having one more attachment devices for coupling operation portion 841 to handle 112 of medical device 110. For example, operation portion 841 may include a flexible clip 805b similar to clip 205b configured to clip onto sides of port 140. Additionally or alternatively, operation portion 841 may include straps, bands, jaws, or pincers, as discussed above for system 200, to secure operation portion 841 to handle 112.

As mentioned above, operation portion 841 may include first actuation mechanism 860 and second actuation mechanism 896 coupled to the control wire to control aspects of end effector 256. First actuation mechanism 860 may be configured to move the control wire proximally and distally, and second actuation mechanism 896 may be configured to rotate the control wire. Similar to the control wires of FIGS. 6A-6B and 7A-7B, the control wire of FIGS. 8A-8C may also have any noncircular shape.

Actuation mechanism 860 may include a lever 803 (shown in FIG. 8B), e.g., a first actuator, that may be operable to pivot or rotate a pivotable member 817. Lever 803 may have a substantially straight shape and may be disposed on a first side 825a of housing 825. The control wire may be coupled to pivotable member 817 such that movement of pivotable member 817 may cause movement of the control wire. Pivotable member 817 may include a body 833 positioned within a cavity 827 of housing 825. Body 833 may have a substantially L shape. For example, body 833 may include a first arm 833a and a second arm 833b extending away from a pivot point 831, and a length of first arm 833a may be greater than a length of second arm 833b. Body 833 may further include an opening 823 on second arm 833b configured to receive lever 803 and a protrusion 889 at an end of first arm 833a. Body 833 may be pivotable about pivot point 831. Pivot point 831 may include an axle, such as a pin, rivet, or similar structure that may be received within an opening of body 833 (e.g., an opening positioned at a vertex of body 833), such that pivotable member 817 is rotationally/pivotably movable about pivot point 831.

Lever 803 may be pivotable about an axle (not shown) and movable along/through a slot (not shown) formed on an outer surface of housing 825. For example, the slot may be similar to slot 215 of FIGS. 2A-2D, except positioned closer to first side 825a of housing 825, and the slot of device 801 may be curved. Lever 803 may be moved (e.g., along an arcuate or straight path) to move the control wire proximally or distally. Movement of lever 803 in a first direction may pivot pivotable member 817 about pivot point 831 such that first arm 833a may move away from a distal end 895 of housing 825, and thus pulling the control wire proximally. Movement of lever 803 in a second direction may pivot pivotable member 817 about pivot point 831 such that first arm 833a may move towards distal end 895 of housing 825, and thus moving the control wire distally.

As mentioned above, operation portion 841 may include second actuation mechanism 896 for rotating the control wire. Second actuation mechanism 896 may include a knob 884 (e.g., a second actuator) disposed on first side 825a of housing 825 and distal to lever 803. Knob 884 may be oriented on/within housing 825, such that a rotational axis M of knob 884 may be approximately parallel to a longitudinal axis of housing 825. An entire circumference of knob 884 may include a plurality of indentations 873 or other gripping features that may assist an operator with rotating knob 884.

Second actuation mechanism 896 may further include first and second wheels 872a, 872b disposed within housing 825 and on opposite sides of housing 825. First wheel 872a may be rotatably fixed to knob 884. For example, first wheel 872a may be attached to a proximal wall/surface of knob 884. Rotation of knob 884 about rotation axis M in a direction (e.g., clockwise or counterclockwise) may rotate first wheel 872a about rotation axis M in the same direction. Second wheel 872b may be aligned with first wheel 872a such that a rotational axis N of second wheel 872b may be parallel to rotational axis M of knob 884/first wheel 872a. Second wheel 872b may also be aligned axially with first wheel 872a. As shown in FIGS. 8B and 8C, first wheel 872a and second wheel 872b may be coupled to one another via belt 878 extending from first wheel 872a to second wheel 872b, such that rotation of knob 884/first wheel 872a about rotation axis M in a direction (e.g., clockwise or counterclockwise) rotates second wheel 872b about rotation axis N in the same direction. A diameter of first wheel 872a may be greater than a diameter of second wheel 872b. Alternatively, a diameter of first wheel 872a may have a same diameter as second wheel 872b, or second wheel 872b may have a greater diameter than first wheel 872a.

FIG. 8C illustrates a top view of features of actuation mechanism 896 (e.g., a top portion of housing 825 removed to expose features of actuation mechanism 896). The control wire may extend distally from pivotable member 817 and through a central opening 870 of second wheel 872b. As mentioned above, the control wire may have any noncircular shape. A size and a shape of central opening 870 of second wheel 872b may be keyed to/complementary to a size and shape of the control wire, which may allow for the control wire to move proximally or distally through central opening 870 of second wheel 872b, but may inhibit rotation of the control wire relative to second wheel 872b. For example, rotation of second wheel 872b about rotation axis N in a direction (e.g., clockwise or counterclockwise) rotates the control wire about rotation axis N in the same direction.

During an exemplary medical procedure, an operator may rotate knob 864/first wheel 872a clockwise about rotational axis M, which in turn rotates second wheel 872b and the control wire clockwise about rotational axis N. Rotation of second wheel 872b and the control wire clockwise about rotational axis N may rotate end effector 256 to a first position. An operator may then rotate knob 864/first wheel 872a counterclockwise about rotational axis M, which in turn rotates second wheel 872b and the control wire counterclockwise about rotational axis N. Rotation of second wheel 872b and the control wire counterclockwise about rotational axis N may rotate end effector 256 to a second position. To transition end effector 256 between actuated and unactuated states, the operator may move lever 803 to move the control wire proximally or distally.

FIG. 9 illustrates features of an exemplary second actuation mechanism 996 that may be used with accessory device 801, e.g., in place of second actuation mechanism 896. Second actuation mechanism 996 may also be used with other aspects disclosed herein. For example, the control wire may be coupled to first actuation mechanism 860 for controlling proximal/distal movement of the control wire and second actuation mechanism 996 for controlling rotation of the control wire. Actuation mechanism 996 may include a gear assembly 999 coupled to a knob (not shown) similar to knob 884. In this example, the knob and gear assembly 999 may be positioned on a second side 825b of housing 825, opposite first side 825a. Gear assembly 999 may include a first, outer gear 904 having teeth 978 (e.g., internal teeth) extending radially inward towards a rotational axis of outer gear 904. Outer gear 904 may be rotatably fixed to the knob. Gear assembly 999 may further include a second, inner gear 906 having teeth 980 (e.g., external teeth) extending radially outward, away from a rotational axis of inner gear 906. Teeth 978 of outer gear 904 may be configured to engage with teeth 980 of inner gear 906 to rotate inner gear 906. Inner gear 906 may include a central opening 974 having a noncircular shape (e.g., a rectangle shape) for receiving the control wire having a complementary, noncircular shape. As such, the control wire may be rotatably fixed to inner gear 906. Rotation of the knob/outer gear 904 in a direction (e.g., clockwise or counterclockwise) rotates inner gear 906 and the control wire/end effector 256 in a same direction.

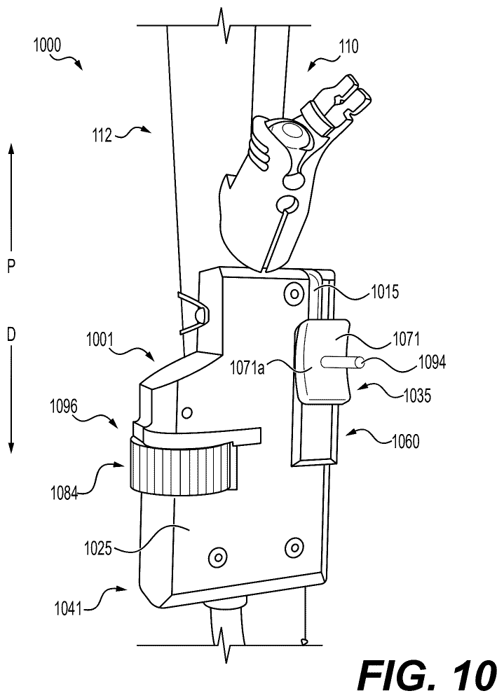

FIG. 10 illustrates an exemplary medical system 1000 that includes medical device 110 coupled to an exemplary accessory device 1001. Accessory device 1001 may have any of the features of accessory devices 201, 501, 601, 701, 801, unless otherwise specified herein. Accessory device 1001 may include a port assembly (not shown) having any of the features of port assemblies 207, 507, 607, 707, 807. The port assembly may be configured to couple to an insertion portion (not shown) having any of the features of insertion portions 250, 550. For example, the insertion portion of accessory device 1001 may include a shaft (not shown) extending distally from the port assembly and a distal end of the shaft may include an end effector (not shown), e.g., end effector 256. A control wire (not shown but having any of the features of control wires 281, 581) may extend proximally from end effector 256, through the shaft, through port assembly, and to an operation portion 1041 of accessory device 1001. The control wire may be coupled to a first actuation mechanism 1060 and a second actuation mechanism 1096. Second actuation mechanism 1096 may have any of the features of actuation mechanism 896. For example, second actuation mechanism 1096 may include a knob 1084 having any of the features of knob 884 for controlling rotation of the control wire/end effector 256.

First actuation mechanism 1060 may be configured to control proximal/distal movement of the control wire to transition end effector 256 between actuated and unactuated states. First actuation mechanism 1060 may include a slider 1035 movable along a slot 1015 formed on a front side of housing 1025. Slot 1015 may be a linear slot extending approximately parallel to a longitudinal axis of housing 1025 and/or handle 112. Slider 1035 may include a contact element 1071 similar to contact element 671 at a proximal portion of slider 1035. Contact element 1071 may include a concave surface 1071a.

An optional post 1094 may extend from a bottom of concave surface 1071a and approximately perpendicular to a longitudinal axis of housing 1025. Post 1094 may assist an operator with moving slider 1035 along slot 1015. For example, an operator may push downward on post 1094 to move slider distally along slot 1015 to move the control wire distally. For example, the operator may push upward on post 1094 to move slider proximally along slot 1015 to move the control wire proximally. In some examples, post 1094 may be integral with remaining portions of slider 1035. In other examples, post 1094 may a detachable/attachable piece that may be received within an opening formed on concave surface 1071a.

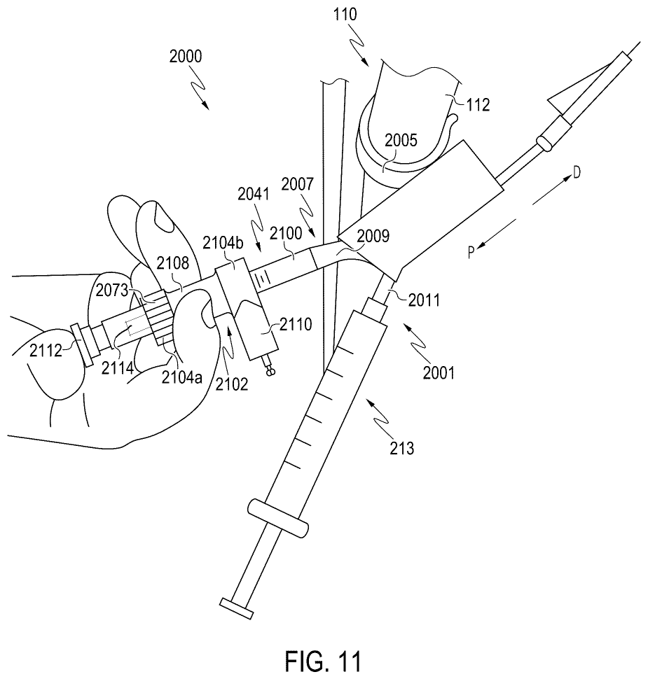

FIG. 11 illustrates an exemplary medical system 2000 that includes an exemplary accessory device 2001 coupled to medical device 110. Accessory device 2001 may include one or more attachment devices for coupling accessory device 2001 to handle 112 of medical device 110. For example, accessory device 2001 may include a clip 2005 configured to couple to handle 112 of medical device 110. Additionally or alternatively, accessory device 2001 may include straps, bands, jaws, and/or pincers, as discussed above for system 200, to secure accessory device 2001 to handle 112 of medical device 110.

Accessory device 2001 may include an operation portion 2041 coupled to a port assembly 2007 having any of the features of port assemblies 207, 507, 607, 707, 807. For example, port assembly 2007 may include a first port 2009 configured to couple to a main body 2100 of operation portion 2041 such that first port 2009 may be in fluid communication with one or more cavities and/or slots of main body 2100. First port 2009 may secure port assembly 2007 to operation portion 2041, while allowing main body 2100 to rotate relative to first port 2009. Port assembly 2007 may include a second port 2011 configured to couple to a syringe 213 or other fluid source for delivering fluid to a target site. Although port assembly 2007 is illustrated as having a Y-shape, port assembly 2007 may alternatively have a T-shape.

Port assembly 2007 may be configured to couple an insertion portion (not shown) having any of the features of insertion portions 250, 550. For example, the insertion portion of accessory device 2001 may include a shaft (not shown) extending distally from port assembly 2007 and a distal end of the shaft may include an end effector (not shown), e.g., end effector 256. A control wire (not shown but having any of the properties of control wires 281, 581) may extend proximally from end effector 256, through the shaft, through first port 2009, and into a cavity and/or slot 2114 of main body 2100.

A proximal end of the control wire may be coupled to an internal connector of actuator 2102 (e.g., a knob, a spool) of operation portion 2041, e.g., an internal connector within second flange portion 2104b of actuator 2102, to secure the control wire and actuator 2102 to main body 2100. Actuator 2102 may be configured to move in the proximal/distal directions (e.g., an axial direction) along slot 2114 to move the control wire in the proximal/distal directions. As mentioned above, main body 2100 may be configured to rotate relative to first port 2009. Because the control wire is secured to main body 2100, rotation of main body 2100 relative to first port 2009 rotates the control wire. Actuator 2102 may rotate with main body 2100 and thus may be used to rotate the control wire.

Actuator 2102 may include a first flange portion 2104a, a second flange portion 2104b, and a central portion 2108 (e.g., an indented portion) extending between first and second flange portions 2104a, 2104b. A diameter of each of first and second flange portions 2104a, 2104b may be greater than a diameter of central portion 2108. First flange portion 2104a may include a plurality of indentations 2073 around an entire circumference of flange portion 2104a. Indentations 2073 may assist an operator with gripping first flange portion 2104a to rotate actuator 2102/main body 2100 and/or serve as markers to indicate to an operator that actuator 2102 is rotatable. Additionally or alternatively, first flange portion 2104a may include any combination of gripping features and/or rotational markers, e.g., bumps, flat surfaces, recesses, etc.

Actuator 2102 may include a connection portion 2110 having a plug coupled to the control wire. The plug may be configured to couple to an electrical supply to electrify the control wire, which may provide, for example, electricity to end effector 256 for electrical cautery or cutting of tissue. As illustrated in FIG. 11, connection portion 2110 may extend from second flange portion 2104b perpendicularly to a central longitudinal axis of actuator 2102. Alternatively, connection portion 2110 may extend from second flange portion 2104b transversely to a central longitudinal axis of actuator 2102. In another alternative, connection portion 2110 may be omitted from actuator 2102 and/or may be disposed on any portion of main body 2100.

Operation portion 2041 may further include a proximal body 2112 coupled to a proximal end of main body 2100. Proximal body 2112 may be fixed to main body 2100 such that proximal body 2112 and main body 2100 may rotate together. In some aspects, as illustrated in FIG. 11, proximal body 2112 may serve as a thumb rest when an operator is using other finger(s) to move actuator 2102 proximally or distally along slot 2114. Additionally or alternatively, an operator may place their thumb and their index finger on proximal body 2112 to rotate proximal body 2112/main body 2100 relative to first port 2009 and thus rotate the control wire/end effector 256. Similar to actuator 2102, proximal body 2112 may include any combination of gripping features and/or rotational markers, e.g., bumps, flat surfaces, recesses, etc.

In use, an operator may place central portion 2108 of actuator 2102 in between their index finger and middle finger, and may place their thumb on proximal body 2112, as shown in FIG. 11. The operator may use their index finger and middle finger to move actuator 2102 proximally along slot 2114 to transition the control wire to a pulled/tensioned state (e.g., a proximal configuration). In the proximal configuration, in an example in which end effector 256 is a tome, end effector 256 may be in an actuated state (e.g., a forward bend), as shown in FIG. 3A. In other examples, the proximal configuration may correspond to actuated states of other types of end effectors 256 or may correspond to an unactuated state of other types of end effectors 256.

To transition the control wire to a relaxed state (e.g., a distal configuration), the operator may move actuator 2102 distally along slot 2114. In the distal configuration, in an example in which end effector 256 is a tome, end effector 256 may be in an unactuated or relaxed state, as shown in FIG. 3B. In other examples, the second configuration may correspond to unactuated states of other types of end effectors 256 or may correspond to an actuated state of other types of end effectors 256. To control rotation of end effector 256, the operator may place their thumb and index finger on first flange portion 2104a to rotate actuator 2102/main body 2100/proximal body 2112 relative to first port 2009 or the operator may place their thumb and index finger on proximal body 2112 to rotate actuator 2102/main body 2100/proximal body 2112 relative to first port 2009.

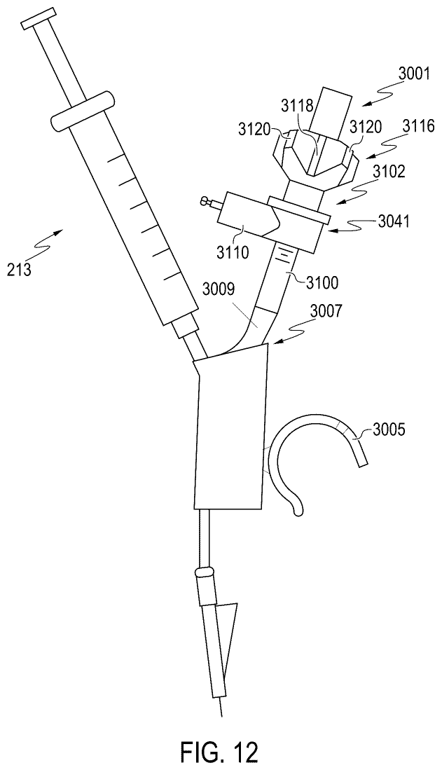

FIG. 12 illustrates an exemplary accessory device 3001 configured to couple to medical device 110. Accessory device 3001 may have any of the features of accessory device 2001, unless otherwise specified herein. Accessory device 3001 may include an attachment device 3005, a port assembly 3007, an operation portion 3041 having a body 3100, and an insertion portion (not shown) having any of the features of insertion portions 250, 550. Accessory device 3001 may optionally be coupled to syringe 213 or other fluid source. Similar to accessory device 2001, a control wire (not shown but having any of the features of control wires 281, 581) may extend proximally from end effector 256 and to an internal connector of an actuator 3102 having any of the features of actuator 2102. Actuator 3102 may be configured to move in the proximal/distal directions along a slot of main body 3100 similar to slot 2114 of main body 2100 to move the control wire in the proximal/distal directions to transition end effector 256 between actuated and unactuated states. Actuator 3102 may be further configured to rotate with main body 3100 relative to a first port 3009 of port assembly 3007 to rotate the control wire/end effector 256. Actuator 3102 may optionally include a connection portion 3110 similar to connection portion 2110.

In this example, actuator 3102 may include a contact element 3116 at a proximal portion of actuator 3102. Contact element 3116 may include alternating recesses 3118 and ridges 3120. For example, contact element 3116 may be substantially star-shaped having pointed recesses 3118/ridges 3120. In other examples, contact element 3116 may have curved recesses 3118/ridges 3120. Contact element 3116 may include any number of recesses 3118 and ridges 3120.

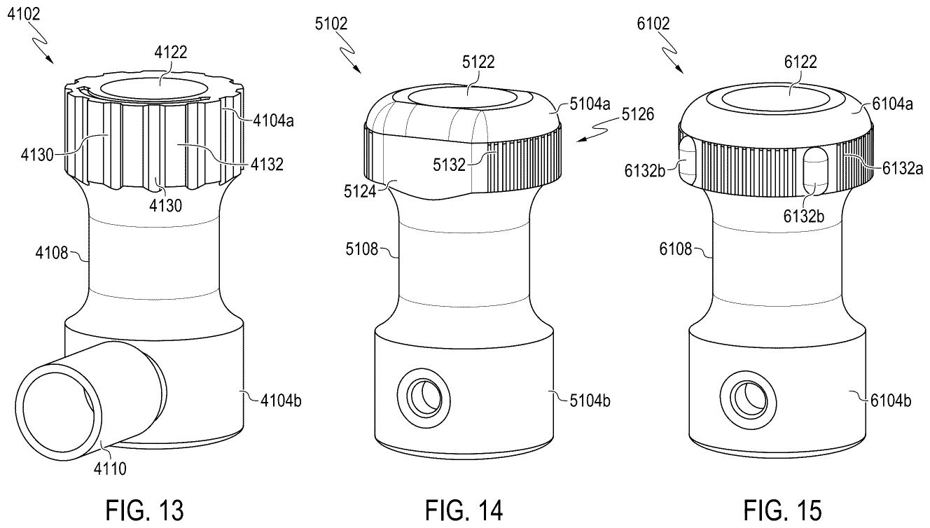

FIGS. 13-15 illustrate exemplary actuators 4102, 5102, 6102 that may be used with main bodies 2100, 3100 of accessory devices 2001, 3001 (e.g., in place of actuators 2102, 3102). However, it will be appreciated that the features of actuators 2102, 3102, 4102, 5102, 6102 described herein may be combined in any way to e.g., improve actuator ergonomics and usability, serve as markers to an operator that the actuator is rotatable in addition to being movable in the proximal/distal directions, and/or serve as rotation markers, e.g., to assist an operator track how much main bodies 2100, 3100 have been rotated.

FIG. 13 illustrates exemplary actuator 4102 having any of the features of actuators 2102, 3102, unless otherwise specified herein. Actuator 4102 may include a first flange portion 4104a, a second flange portion 4104b, and a central portion 4108 (e.g., an indented portion) extending between first and second flange portions 4104a, 4104b. Actuator 4102 may include a connection portion 4110 similar to connection portions 2110, 3110. Actuator 4102 may include an opening 4122 for receiving main bodies 2100, 3100.

Central portion 4108 and second flange portion 4104b may include smooth, outer surfaces. First flange portion 4104a may include alternating protrusions 4132 and indentations 4130 disposed around an entire circumference of first flange portion 4104a. Each protrusions 4132 and each indentation 4130 may extend parallel to a central longitudinal axis of actuator 4102. An outer surface of each indentation 4130 may be substantially flat or substantially pointed. Although second flange portion 4104b is illustrated as having a smooth surface, second flange portion 4104b may alternatively include alternating protrusions 4132 and indentations 4130.

FIG. 14 illustrates exemplary actuator 5102 having any of the features of actuators 2102, 3102, 4102, unless otherwise specified herein. Actuator 5102 may include a first flange portion 5104a, a second flange portion 5104b, and a central portion 5108 (e.g., an indented portion) extending between first and second flange portions 5104a, 5104b. Actuator 5102 may include an opening 5122 for receiving main bodies 2100, 3100.

A connection portion, such as connection portions 2110, 4110, may be omitted or may be present (but not shown). Central portion 5108 and second flange portion 5104b may include smooth, outer surfaces. First flange portion 5104a may include one or more flat sections 5124 and one or more curved sections 5126. As illustrated in FIG. 14, first flange portion 5104a may include two flat sections 5124 disposed on opposite sides of actuator 5102 and two curved sections 5126 disposed on opposite sides of actuator 5102. Each flat section 5124 may include a planar surface and each curved section 5126 may include a curved surface. The curved surface of curved section 5126 may include a plurality of protrusions 5132 extending radially outward from the curved surface. Protrusions 5132 may extend between a distalmost edge of first flange portion 5104a and a proximal, rounded edge of first flange portion 5104a. For example, protrusions 5132 may terminate distal to the proximal, rounded edge of first flange portion 5104a. Although second flange portion 5104b is illustrated as having a smooth surface, second flange portion 5104b may alternatively include one or more flat sections 5124 and/or one or more curved sections 5126.

FIG. 15 illustrates exemplary actuator 6102 having any of the features of actuators 2102, 3102, 4102, 5102, unless otherwise specified herein. Actuator 6102 may include a first flange portion 6104a, a second flange portion 6104b, and a central portion 6108 (e.g., an indented portion) extending between first and second flange portions 6104a, 6104b. Actuator 6102 may include an opening 6122 for receiving main bodies 2100, 3100.

A connection portion, such as connection portions 2110, 4110, may be omitted or may be present (but not shown). Central portion 6108 and second flange portion 6104b may include smooth, outer surfaces. First flange portion 6104a may include alternating sets of thin, linear protrusions 6132a and protrusions 6132b having an elongated, dome shape. For example, a width of protrusion 6132b may be greater than a width of protrusion 6132a. Protrusions 6132a, 6132b may extend radially outward from an outer surface of first flange portion 6104a. Protrusions 6132a, 6132b, may extend approximately parallel to a central longitudinal axis of actuator 6102 and may be arranged around an entire circumference of first flange portion 6104a. Protrusions 6132a, 6132b may extend between a distalmost edge of first flange portion 6104a and a proximal, rounded edge of first flange portion 6104a. For example, protrusions 6132a, 6132b may terminate distal to the proximal, rounded edge of first flange portion 6104a. Although second flange portion 6104b is illustrated as having a smooth surface, second flange portion 6104b may alternatively include sets of thin, linear protrusions 6132a and/or protrusions 6132b.

In some examples, protrusions 6132b may align with features of main bodies 2100, 3100 to indicate to an operator how much main bodies 2100, 3100 have been rotated relative to first port 2009, 3009, respectively. In an example in which actuator 6102 is coupled to main body 2100, protrusions 6132b may align with detents (not shown) at an intersection (e.g., a connection point) of main body 2100 and first port 2009. The intersection of main body 2100 and first port 2009 may include two detents spaced 180 degrees apart and configured to engage with respective slots to lock a position of main body 2100 relative to first port 2009. Aligning protrusions 6132b with the detents may assist an operator keep track of how much main body 2100 has been rotated relative to first port 2009, e.g., 90 degrees, 180 degrees, etc. It will be appreciated that actuators 2102, 3102, 4102, 5102 may include protrusions 6132b and/or other rotational markers.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical device comprising:
a shaft extending from a proximal end to a distal end, wherein a control wire extends through a lumen of the shaft and to an end effector at the distal end of the shaft; and
an operation portion coupled to the shaft, the operation portion including:
a housing;
one or more attachment devices for coupling the medical device to another medical device; and
an actuation mechanism for actuating the end effector, wherein the actuation mechanism includes a lever extending through an opening of a pivotable body within the housing, and wherein the control wire is coupled to the pivotable body.

2. The medical device of claim 1, wherein the medical device further includes a port assembly configured to couple the shaft to the operation portion, and wherein the port assembly is rotatable relative to the operation portion.

3. The medical device of claim 2, wherein the port assembly includes two channels in fluid communication with the lumen of the shaft, and wherein one channel of the two channels is configured to receive the control wire.

4. The medical device of claim 3, wherein another channel of the two channels is configured to couple to a fluid source.

5. The medical device of claim 3, wherein the pivotable body includes a protrusion configured to align with the one channel of the two channels.

6. The medical device of claim 1, wherein the housing includes a first slot on an outer surface of the housing and a second slot inside the housing, and wherein the lever extends through the first slot and the second slot.

7. The medical device of claim 6, wherein the lever is movable along the first slot and the second slot, and relative to the housing.

8. The medical device of claim 6, wherein the first slot and the second slot extend parallel to one another.

9. The medical device of claim 1, wherein the pivotable body includes a channel configured to receive the control wire.

10. The medical device of claim 9, wherein the channel includes a first portion and a second portion extending approximately parallel to one another, and wherein the channel includes a third portion extending between and approximately perpendicular to the first portion and the second portion.

11. The medical device of claim 10, wherein the pivotable body includes a protrusion, and wherein the second portion of the channel extends along the protrusion.

12. The medical device of claim 9, wherein the control wire is fixed to the channel.

13. The medical device of claim 9, wherein the channel includes a first end and a second end, and wherein a second end of the channel is configured to align with the lumen of the shaft.

14. The medical device of claim 1, wherein the actuation mechanism is a first actuation mechanism configured to move the control wire in a proximal direction or a distal direction, and wherein the medical device include a second actuation mechanism configured to rotate the control wire.

15. The medical device of claim 14, wherein the second actuation mechanism includes a first wheel rotatably coupled to a second wheel, wherein the control wire extends through the first wheel, and wherein the second wheel is coupled to a knob.

16. A medical device comprising:
a shaft, wherein a control wire extends through a lumen of the shaft and to an end effector at a distal end of the shaft; and
an operation portion coupled to the shaft, the operation portion including:
a housing configured to be coupled to a handle of a scope device; and
an actuation mechanism coupled to the control wire for actuating the end effector, wherein the actuation mechanism includes an actuator, a first arm, and a second arm movably coupled to one another.

17. The medical device of claim 16, wherein a first end of the first arm is coupled to the actuator and a second end of the first arm is coupled to a first end of the second arm, and wherein a second end of the second arm is coupled to the control wire.

18. The medical device of claim 16, wherein the first arm is configured to move in an axial direction and the second arm is configured to rotate.

19. A medical device comprising:
a shaft including an end effector at a distal end of the shaft, wherein a control wire extends through the shaft and is coupled to the end effector;
an operation portion including:
a housing configured to couple to a handle of another medical device;
and
an actuation mechanism for actuating the end effector, wherein the actuation mechanism includes a knob, a rod, and a pair of wheels, wherein the rod extends from the knob to a first wheel of the pair of wheels, and wherein the control wire extends through and is rotatably fixed to a second wheel of the pair of wheels.

20. The medical device of claim 19, wherein a rotational axis of the first wheel is transverse to a rotational axis of the second wheel.
